(19) United States
(12) Patent Application Publication
(10) Pub. No.: US 2005/0096716 A1
(43) Pub. Date: May 5, 2005

(54) Device and process for regulating a numerical value for drug-induced muscle relaxation

(75) Inventors: Konrad S. Stadler, Unterehrendingen (CH); Peter M. Schumacher, Kirchlindach (CH); Daniel Leibundgut, Bremgarten (CH)

Correspondence Address: MCGLEW & TUTTLE, PC, 1 SCARBOROUGH STATION PLAZA, SCARBOROUGH, NY, 10510-0827, US

(73) Assignee: Drager Medical AG & Co., Lubeck (DE)

(21) Appl. No.: 10/941,258

(22) Filed: Sep. 15, 2004

(30) Foreign Application Priority Data
Oct. 30, 2003 (DE) 103 50 764.7

(51) Int. Cl. A61N 1/00

(52) U.S. Cl. 607/116; 607/2

(57) ABSTRACT

A process for regulating a numerical value for the drug-induced muscle relaxation is provided as well as a corresponding device with an infusion pump (5) for administering a muscle-relaxing drug to a patient (1) connected thereto, an electric stimulator (2) connected to the patient (1) via electrodes (7) for stimulating a nerve, a means (8) for measuring the response of the muscle to the stimulation of the nerve and an evaluation and control unit (6) for regulating the response of the muscle. The evaluation and control unit (6) is connected with the infusion pump (5), the stimulator (2) and the means (8). The evaluation and control unit (6) includes a set point transducer (11) for sending a first set point for the response, a first comparison unit (3), which forms a first difference from a first actual value for the response and the first set point, and with a first regulator (9), which forms from the first difference a second set point for e) a second comparison unit (4), which compares this second set point with a second actual value for the stimulation, and with a second regulator (10), which forms a manipulated variable from this for the infusion pump (5), so that the dose of the muscle-relaxing drug is increased by the infusion pump (5) when the second actual value exceeds the second set point, and the dose of the muscle-relaxing drug is reduced by the infusion pump (5) when the second actual value drops below the second set point until the second actual value and the second set point agree.

### CROSS REFERENCE TO RELATED APPLICATIONS

This application claims the benefit of priority under 35 U.S.C. § 119 of DE 103 50 764.7 filed Oct. 30, 2003, the entire contents of which are incorporated herein by reference.

### FIELD OF THE INVENTION

The present invention pertains to a device and a process for regulating a numerical value for drug-induced muscle relaxation in a patient.

### BACKGROUND OF THE INVENTION

Unconsciousness, insensitivity to pain and muscle relaxation are induced, intraoperatively maintained and terminated in an optimal manner for a balanced anesthesia. Drugs with a correspondingly specific action, which are administered in a combination to the patient to be anesthetized, are now available for this. The actions of the drugs are monitored by the anesthesiologist, who observes the patient, as well as by means of a corresponding monitoring.

A device and a process for regulating a numerical value for the patient respiration are known from DE 100 15 026 A1 and corresponding U.S. Pat. No. 6,691,705. An anesthetic gas mixture is dosed there directly according to the patient's needs with a regulatory circuit for regulating a numerical value on the basis of the evaluation of the electroencephalogram.

Besides anesthetics, drugs for muscle relaxation, are used for anesthesia, e.g., during surgery. So-called muscle relaxants or neuromuscular blockers are used in such a case. The patient's intubation is thus facilitated, and stimuli caused by the surgery cannot elicit any unforeseeable motor reactions in the patient. In view of an optimal dosage of the muscle relaxants, the action of these muscle relaxants on muscle relaxation is to be monitored.

The muscle relaxation is usually monitored by means of a stimulating current applied in a pulsed form at a superficial nerve and a subsequent measurement of the corresponding response of the muscle. The stimulation, i.e., the application of the stimulating current, is painful and is performed only when the patient is anesthetized. The response of the muscle can be determined quantitatively by means of an electromechanogram, i.e., by recording the force, by means of electromyography, i.e., the measurement of the electric activity, or by accelerometry, i.e., acceleration measurement. During the measurement of the muscle relaxation in case of nondepolarizing muscle relaxants, stimulation is performed not only with a single stimulus, i.e., a single stimulating current applied in a pulsed form, but mostly with a train of four stimuli. The at least four stimuli are usually applied within two seconds, which are called “train of four” or TOF stimulation for short. The number of responses of the muscle to a TOF stimulation, the so-called TOF counts, decreases with increasing muscle relaxation until there is no response at all. The quantitative degree of muscle relaxation is expressed as the ratio of the first muscular response, the measured value, to a response measured at the beginning of the anesthesia without the use of muscle relaxants, the reference value. This ratio of the measured value to the reference value is called T1% and stated in percent.

Permanent monitoring of the muscle relaxation and the automatically regulated administration of the muscle relaxants proved to be very meaningful for the optimal dosing of muscle relaxants, especially short-acting muscle relaxants. The reason for this is the great differences in the quantity of muscle relaxants needed as a function of the individual patient and depending on the point in time during the surgery. The dose can be minimized and the desired effect can be reached with the automatic regulation of the administration. The aftereffects and side effects of a drug-induced muscle relaxation and the duration of the spontaneous reversion of the muscular blocking are thus also minimized, so that it is not necessary to additionally perform a reversion with drugs. The determination of T1% cannot be performed routinely in clinical practice, partly because of an undesired prolongation of the induction by a stabilization time of 10 to 20 minutes, and partly because of a frequently necessary manual artificial respiration of the patient during this time. Prolongation of the induction makes anesthesia needlessly expensive, and manual artificial respiration represents an additional risk for the patient.

### SUMMARY OF THE INVENTION

The object of the present invention is to present a device and a process for regulating a numerical value for the drug-induced muscle relaxation, with which the muscle relaxation is regulated reliably and in a simple way during anesthesia.

According to the invention, a device is provided with a control circuit for regulating a numerical value for a drug-induced muscle relaxation. The device includes an infusion pump for administering a muscle-relaxing drug to a patient connected thereto. An electric stimulator is connected to the patient via electrodes for stimulating a nerve. A means measures the response of the muscle to the stimulation of the nerve. An evaluating and control unit regulates the response of the muscle. The evaluating and control unit is connected with the infusion pump, the stimulator and the means. The evaluating and control unit comprises a set point transducer for sending a first set point for the response, a first comparison unit, which forms a first difference from a first actual value for the response and the first set point, and with a first regulator, which forms from the first difference a second set point. A second comparison unit compares this second set point with a second actual value for the stimulation. A second regulator forms a manipulated variable from this for the infusion pump so that the dose of the muscle-relaxing drug is increased by the infusion pump when the second actual value exceeds the second set point and the dose of the muscle-relaxing drug is reduced by the infusion pump when the second actual value drops below the second set point until the second actual value and the second set point agree.

According to another aspect of the invention, a process is provided for regulating a numerical value for the drug-induced muscle relaxation. A muscle-relaxing drug is administered by an infusion pump to a patient connected thereto. A nerve is stimulated by an electric stimulator connected to the patient via electrodes. A means measures the response of the muscle to the stimulation of the nerve. An evaluating and control unit regulates the response of the muscle, wherein a set point transducer sends a first set point for the response, a first comparison unit forms a first difference from a first actual value for the response and the first set point, and a first regulator forms from the first difference a second set point. A second comparison unit compares this second set point with a second actual value for the stimulation, and a second regulator forms from this a manipulated variable for the infusion pump so that the dose of the muscle-relaxing drug is increased by the infusion pump when the second actual value exceeds the second set point, and the dose of the muscle-relaxing drug is reduced by the infusion pump when the second actual value drops below the second set point until the second actual value and the second set point agree.

The present invention is a clinical solution suitable for practical use. The device according to the present invention has the structure of a cascaded control circuit. For example, a value for T1% is regulated in the inner cascade of the control circuit, and the number of responses of the muscle to a TOF stimulation, the so-called TOF counts, are regulated in the outer cascade of the control circuit. A number of responses is preset as the set point for the entire control circuit. This is, as was mentioned, an indicator of the muscle relaxation. The inner control circuit receives a set point for T1%. Known population mean values are selected as the setting at the beginning. The process according to the present invention describes the corresponding cascaded control circuit of the muscle responses to a drug-induced muscle relaxation.

The present invention will be explained below as an example on the basis of the drawings. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which a preferred embodiment of the invention is illustrated.

### BRIEF DESCRIPTION OF THE DRAWINGS

FIG. 1 is a schematic view of a device for regulating a numerical value for the muscle relaxation; and

FIG. 2 is a time curve of the value of the TOF count, the muscle response and the corresponding rate of infusion of a muscle-relaxing drug.

### DESCRIPTION OF THE PREFERRED EMBODIMENT

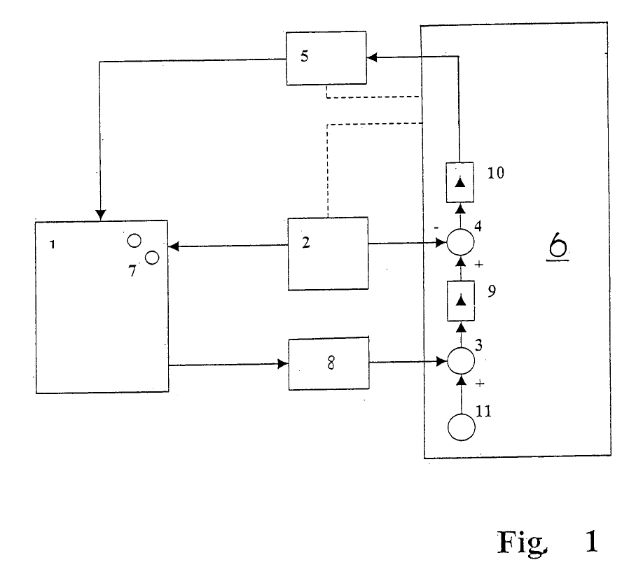

The device for regulating a numerical value for the muscle relaxation, which is shown schematically in FIG. 1, comprises an electric stimulator 2, a means 8 for measuring the response of the muscle, an infusion pump 5 for the muscle-relaxing drug and an evaluation and control unit 6, which receives and evaluates the signals of the means 8 and actuates the stimulator 2 and the infusion pump 5. The actuation of the stimulator 2 and the infusion pump 5 by the evaluation and control unit 6 is indicated by dashed connection lines.

The muscle-relaxing drug is infused by means of the infusion pump 5 via a venous access in a patient 1.

To evoke a muscle response, a TOF stimulation is applied to a corresponding nerve by the stimulator 2. The responses of the muscle are measured by means of the means 8. The evaluation and control unit 6 comprises a unit for regulating the numerical value, comprising a set point transducer 11, a first comparison unit 3 and a first regulator 9.

The evaluation and control unit 6 comprises, furthermore, a control circuit, which is subordinated (as co-working controls) to the control circuit for regulating the numerical value, for regulating the muscle response in percentage compared with the muscle response in the nonrelaxed state, i.e., for regulating the T1% value, in the manner of a cascade circuit. The subordinated control circuit comprises a second comparison unit 4 and a second regulator 10.

Sensors for recording a mechanogram or an electromyogram are preferably used to measure the muscle response by means of the means 8, but the use of an accelerometric measurement of the muscle response is possible as well.

The TOF stimulation, which is applied by the stimulator 2, comprises four individual current pulses with a maximum current intensity of 70 mA. The means 8 measures the number and the intensity of the muscle responses and compares the intensity of the first muscle response with a reference measurement performed at the beginning, so that a percentage response T1% of the muscle is obtained. Four responses of the muscle, i.e., four TOF counts, are measured in the nonrelaxed state of the patient 1, and 100% is set for the percentage response of the muscle. No muscle responses are detected any longer in case of complete muscle relaxation, and the percentage response is zero.

During the operation of the device, a TOF stimulation is applied by the stimulator 2, e.g., to the ulnar nerve. The response thus evoked of the adductor pollicis muscle are recorded by means of the means 8 and evaluated by the evaluation and control unit 6. The numerical value, comprising the number of muscle responses, the TOF counts, is sent as an actual value to the first comparison unit 3. By means of the set point transducer 11, the user presets a number 1 of muscle responses as a numerical value, which is sent as a set point to the first comparison unit 3. The difference between the actual value and the set point is formed at the first comparison unit 3, and the first regulator 9 forms a set point for the second comparison unit 4 for the percentage response of the muscle from this difference. The measured percentage response of the muscle is sent as an actual value to the second comparison unit 4. The second regulator 10 determines from the difference formed at the second comparison unit 4 a rate of infusion, which is sent as a manipulated variable to the infusion pump 5 for the muscle-relaxing drug. When the set point for the numerical value is exceeded by the measured value, the dosage of the drug increases as a consequence via the infusion pump 5, and the dosage is reduced if the actual value drops below the set point. The quantity dosed by the infusion pump 5 is varied by the two regulators 9, 10 until the numerical value corresponds to the value desired by the user. A closed control circuit is thus obtained for the numerical value by the variation of the quantity infused and the evaluation of the muscle response in patient 1.

As was described above, a control circuit for regulating the percentage response of the muscle is subordinated to the control circuit for regulating the numerical value. With the invention, a more precise control becomes possible compared with the coarse resolution, when using the numerical value alone as the control parameter.

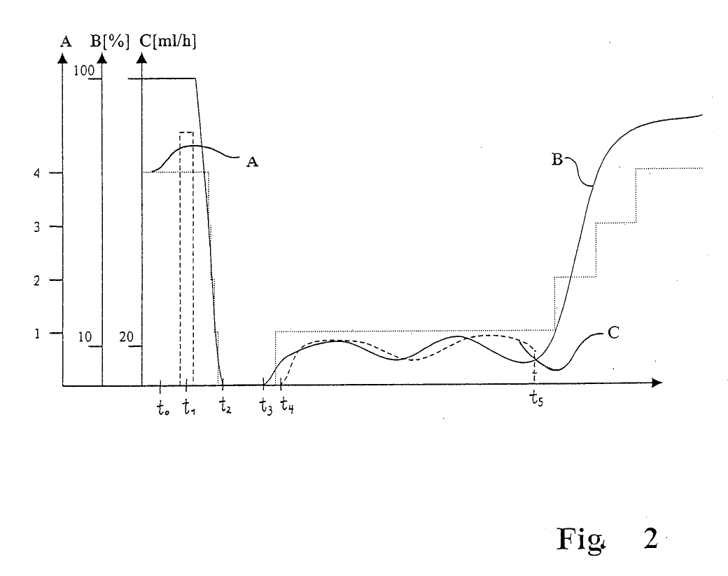

FIG. 2 shows the changes over time on the horizontal axis, showing the integer values for the TOF count, the muscle response in percentage, abbreviated as %, and the corresponding rate of infusion of a muscle-relaxing drug in mL per hour.

The value for the TOF count is represented by the dotted line A, the value T1% by the solid black line B, and the corresponding rate of infusion is illustrated by the dashed line C.

The muscle is not yet relaxed at the time t₀, and the values for the TOF count and T1% are maximal. A bolus dose of the muscle-relaxing drug is administered at the time t₁. This leads to the reduction of the values for the TOF count and T1% to zero during the period [t₂, t₃]. The values for T1% are subsequently maintained by the lowest possible dosage of the muscle-relaxing drug at an optimally low level during the period [t₄, t₅], i.e., the values for the TOF count are maintained at a constant level. At time t₅ at the end of the anesthesia, no muscle-relaxing drug is administered any longer, and the values for the TOF count and T1% tend again toward the original values.

While a specific embodiment of the invention has been shown and described in detail to illustrate the application of the principles of the invention, it will be understood that the invention may be embodied otherwise without departing from such principles.

1. A device with a control circuit for regulating a numerical value for the drug-induced muscle relaxation, the device comprising:
an infusion pump for administering a muscle-relaxing drug to a patient connected thereto;
an electric stimulator connected to the patient via electrodes for stimulating a nerve;
a means for measuring the response of the muscle to the stimulation of the nerve;
an evaluation and control unit for regulating the response of the muscle, which is connected with the infusion pump, the stimulator and the means, the evaluation and control unit comprising a set point transducer for sending a first set point for the response, a first comparison unit, which forms a first difference from a first actual value for the response and the first set point, and with a first regulator, which forms from the first difference a second set point, a second comparison unit, which compares this second set point with a second actual value for the stimulation, and with a second regulator, which forms a manipulated variable from this for the infusion pump so that the dose of the muscle-relaxing drug is increased by the infusion pump when the second actual value exceeds the second set point and the dose of the muscle-relaxing drug is reduced by the infusion pump when the second actual value drops below the second set point until the second actual value and the second set point agree.

2. A device in accordance with claim 1, wherein the stimulation is a so-called “train of four” stimulation.

3. A process for regulating a numerical value for the drug-induced muscle relaxation, the process comprising the steps of:
administrating a muscle-relaxing drug by an infusion pump to a patient connected thereto;
stimulating a nerve by an electric stimulator connected to the patient via electrodes;
using a means to measure a response of the muscle to the stimulation of the nerve;
regulating a response of the muscle using an evaluation and control unit, wherein a set point transducer sends a first set point for the response, a first comparison unit forms a first difference from a first actual value for the response and the first set point, a first regulator forms from the first difference a second set point, a second comparison unit compares this second set point with a second actual value for the stimulation, and a second regulator forms from this a manipulated variable for the infusion pump so that the dose of the muscle-relaxing drug is increased by the infusion pump when the second actual value exceeds the second set point and the dose of the muscle-relaxing drug is reduced by the infusion pump when the second actual value drops below the second set point until the second actual value and the second set point agree.
